(19) United States
(12) Patent Application Publication
(10) Pub. No.: US 2009/0155918 A1
(43) Pub. Date: Jun. 18, 2009

(54) METHOD AND APPARATUS FOR MONITORING BIOMETRICAL DATA

(75) Inventors: Sebastien Payen, Berkeley, CA (US); David C. Walther, Oakland, CA (US); Albert P. Pisano, Danville, CA (US)

Correspondence Address: Trellis Intellectual Property Law Group, PC, 1900 EMBARCADERO ROAD, SUITE 109, PALO ALTO, CA, 94303, US

(73) Assignee: THE REGENTS OF THE UNIVERSITY OF CALIFORNIA, Oakland, CA (US)

(21) Appl. No.: 11/911,952

(22) PCT Filed: Apr. 17, 2006
(86) PCT No.: PCT/US06/14520

§ 371 Date: Nov. 24, 2008

Related U.S. Application Data
(60) Provisional application No. 60/672,749, filed on Apr. 18, 2005.

(51) Int. Cl. G01N 27/00; G01N 33/00

(52) U.S. Cl. 436/149; 436/163; 422/82.01

(57) ABSTRACT

A method and apparatus suitable for monitoring biometrical data (e.g., pH level of a grape) of living organisms is disclosed. A method for monitoring biometrical data includes providing a chemical matter having a chemical agent, contacting the chemical matter with a biological matter, detecting a change in the chemical matter to produce a signal, altering the signal into an electrical signal, and obtaining biometrical data from the electrical signal. An apparatus for determining biometrics data of a living organism includes a capillary tube configured to receive fluid from the living organism, a hydrogel solution having a volume responsive to one or more characteristics of the fluid, a capacitor having at least one plate responsive to the volume of the hydrogel solution, and an inductor coupled to the capacitor. The inductor and the capacitor form a circuit having a resonant frequency. Embodiments of the present invention can advantageously provide a wireless and powerless device for determining biometrics data of living organisms.

### CROSS-REFERENCE TO RELATED APPLICATIONS

This patent application is related to Provisional Patent Application having application No. 60/672,749, filed Apr. 18, 2005, and fully incorporated herein by reference thereto as if repeated verbatim immediately herein. Benefit of the Apr. 18, 2005 filing date for the Provisional Patent Application is claimed.

### FIELD OF THE INVENTION

Embodiments of the present invention are related to a method and apparatus for monitoring biometrical data. More specifically, embodiments of the present invention provide a method and an apparatus for determining a change in a chemical characteristic of a biological matter. More specifically further, embodiments of the present invention provide a method and apparatus for obtaining data (e.g., biometrical data) of a subject, such as a biological matter (e.g., a fruit, a human, etc).

### BACKGROUND OF THE INVENTION

Wireless biosensors capable of monitoring biometrics data of a living organism have long been a goal of biomedical device technology. Functionalized polymers or hydrogels are a well-characterized and developed class of polymer with a high affinity for water (they swell as water diffuses through the polymer fiber matrix) and have been used in some conventional biosensor approaches.

The fabrication of active hydrogel components inside microchannels are known in the art. Due to compatibility with microfabrication techniques, the hydrogel may be fabricated in situ and the stimuli-responsive hydrogels can perform both sensing and actuation functions. Using variation in the geometry of the patterned hydrogel, response times of their valves were improved to less than 10 seconds. However, these devices do not integrate an electronic circuit to detect and measure the chemical signal sensed by the hydrogel.

Several conventional implantable hydrogel-based sensors are known in the art for measuring body analytes such as pH, glucose, urea and penicillin. These describe a biosensor with a hydrogel enclosed in a rigid and biocompatible device. The hydrogel can be functionalized to different type of analytes and swells in response to diffusion of the analytes through the hydrogel medium. The swelling of the hydrogel is then measured with a pressure transducer. A battery powered telemeter engaged to the pressure transducer sends a radio signal to a receiver. This receiver can be attached to a computer or an alarm in order to monitor a patient. However, one drawback of this approach is the requirement of a power source on the device.

Other conventional approaches include a hydrogel actuated and implantable biosensor transducer. This device combines capacitive pressure sensing techniques with biosensitive hydrogel using an adaptable MEMS fabrication platform. However, many features remain to be improved. The biosensor should be miniaturized significantly since the die is 4.3×4.3 mm2 and the capacitor plates are 100 um apart. The hydrogel is an uncrosslinked PHEMA hydrogel which swells in response to calcium nitrate tetrahydrate. In this approach, they utilize the swelling behavior of the hydrogel to power the device. A transduction is performed by a capacitive pressure sensor and the variation in frequency of an RLC tank circuit enables relating the change in concentration of the analyte with the resonance frequency of the tank circuit.

Among the many example applications of biosensor devices are the monitoring (e.g., in California) of humidity in redwood forests. However the devices currently used in such monitoring are usually large and require power or frequent maintenance to change batteries.

What is needed is a device that is relatively inexpensive, powerless and wireless. Such a device could be used much more extensively and allow monitoring of micro-climates in the forests, as an example. In agriculture, such a device could allow monitoring of the growth of grapes destined to wine making or citrus, for example. Furthermore, the hydrogel used for such applications can be designed to examine agricultural virus development (e.g., Pierce disease). Further, this type of device could also be adapted to human monitoring, such as to monitor and provide early alarm indication for at risk patients.

### SUMMARY OF EMBODIMENTS OF THE INVENTION

Embodiments of the invention provide a method and apparatus suitable for monitoring biometrical data of living organisms. Such biometrics data can include the pH level of a grape, for example.

An embodiment of the present invention provides a method for monitoring biometrical data. The method includes providing a chemical matter (e.g., a hydrogel) having a chemical agent (e.g., acrylic acid), contacting the chemical matter with a biological matter (e.g., such as fluid from a grape), detecting a change (e.g., a change in volume) in the chemical matter to produce a signal (e.g., a mechanical signal and/or chemical signal), altering the signal into an electrical signal, and obtaining biometrical data from the electrical signal. The chemical agent is sensitive to a chemical characteristic (e.g., such as pH, glucose, etc) and the biological matter includes the chemical characteristic such that monitoring of biometrical data includes determining a change in the chemical characteristic.

Another embodiment of the present invention provides a method for obtaining data of a subject. The method comprises inserting or immersing a sensor into a subject and retrieving fluid from the subject, transforming a chemical signal from the fluid by using a functionalized polymer, detecting a change in the functionalized polymer, and transmitting an indication of the change in the functionalized swelling polymer. The functionalized polymer can be a swelling polymer (e.g., a hydrogel, etc), by way of example only.

A further embodiment of the present invention provides an apparatus for determining biometrics data of a living organism. The apparatus includes a capillary tube configured to receive fluid from the living organism, a hydrogel solution with a volume responsive to characteristics of the fluid, a capacitor with a plate that moves in response to the volume of the hydrogel solution, and an inductor coupled to the capacitor to form a circuit with a resonant frequency that is related to the analyte through a transduction mechanism.

A further embodiment of the present invention provides for an apparatus to determine the biometrics data of a large sample volume. In this embodiment, the apparatus is immersed in the sample (blood, urine, milk, pressed juice) and data are collected. This embodiment does not require high aspect ratio (ie needle like) capillary tubes, but rather is required to allow the sample to brought into contact with the detection mechanism and the transduction mechanism and data collection are similar to the previous embodiments.

Embodiments of the present invention can advantageously provide a wireless and powerless device for determining biometrics data of living organisms.

A further embodiment of the present invention provides that this apparatus can be fabricated or coupled with other measurement devices to measure change in temperature, light intensity, humidity,

These provisions, together with the various ancillary provisions and features which will become apparent to those skilled in the art as the following description proceeds, are attained by the methods and assemblies of the present invention.

### BRIEF DESCRIPTION OF THE DRAWINGS

FIG. 1 is a view of a biosensor monitoring device in accordance with an embodiment of the present invention.

FIGS. 2-9A are step by step diagrams for producing another embodiment of the hydrogel/capacitors assembly.

FIG. 9B is a top plan view of another embodiment of the device.

FIG. 9C is a vertical sectional view taken in direction of the arrows and along the plane of line 9C-9C in FIG. 9B.

FIG. 9D illustrates a “wafer” process for producing a multiplicity of devices.

FIG. 10 is a vertical sectional view of another embodiment of the hydrogel/capacitors assembly.

FIG. 11 is a diagram of an embodiment of the circuit assembly.

FIG. 12 is a block flow diagram for an embodiment of the signal pathway, along with a diagram of the antenna coupled to the hydrogel/capacitors assembly for detecting a change in capacitance which is directly related to the measurand in the organism fluid.

FIG. 13 is a cross-sectional view of the transduction feature of embodiments of the present invention.

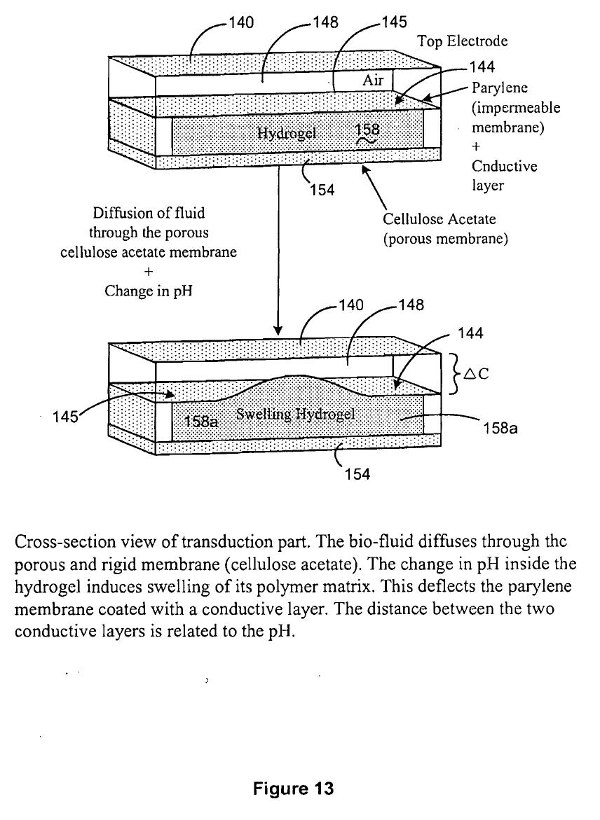

FIG. 14 is a graph of dx/x (change in distance between capacitor plates/initial distance between capacitor plates before the change in distance) vs. pH (i.e., the pH on the x-axis for any particular dx/x on the y-axis).

FIG. 15 is an illustration of an embodiment of the biosensing device after being inserted into a grape.

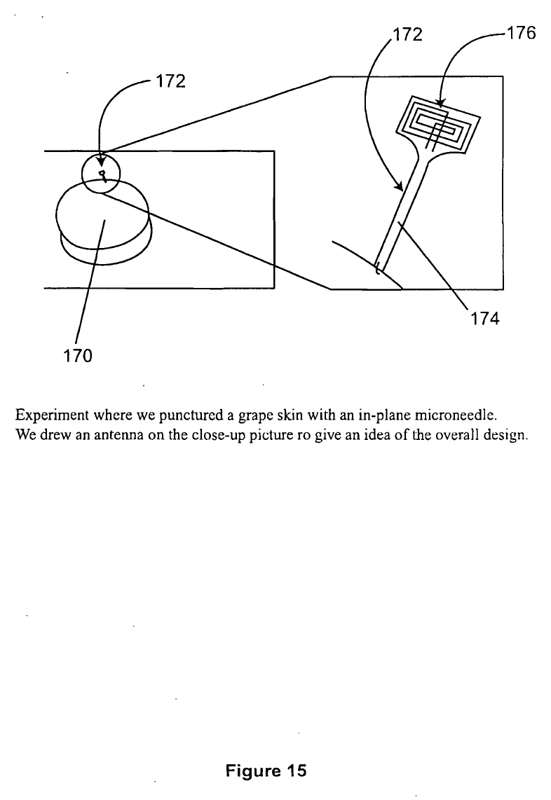

FIG. 16 is a block flow diagram for another embodiment of a signal pathway, illustrating the production of a change in resonance frequency in accordance with the particular measurand in the bio-organism fluid being monitored and/or measured.

### DETAILED DESCRIPTION OF EMBODIMENTS OF THE INVENTION

In the description herein, numerous specific details are provided, such as examples of components and/or methods, to provide a thorough understanding of the embodiments of the present invention. One skilled in the relevant art will recognize, however, that an embodiment of the invention may be practiced without one or more of the specific details, or with other apparatus, systems, assemblies, methods, components, materials, parts, and/or the like. In other instances, well-known structures, materials, or operations are not specifically shown or described in detail to avoid obscuring aspects of the embodiments of the present invention.

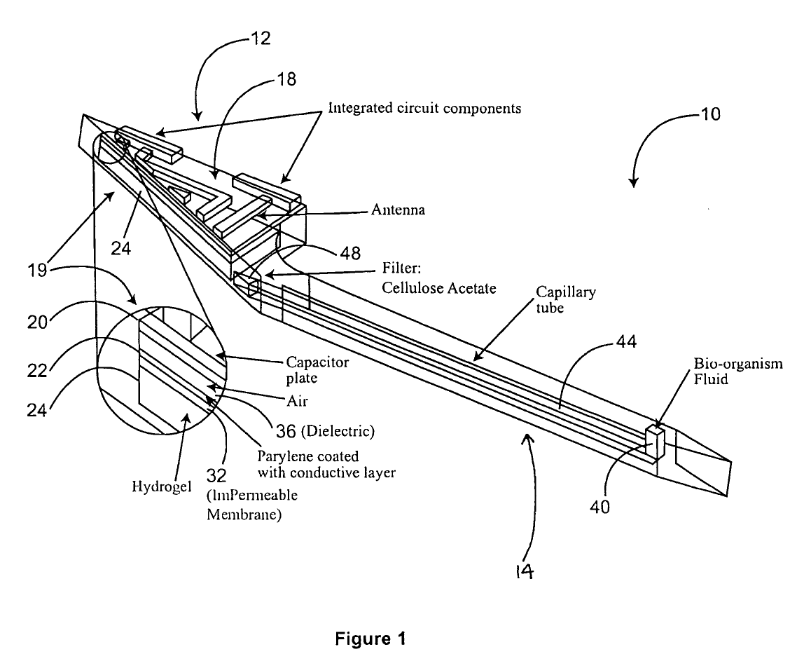

Referring in detail now to the drawings, there is seen in FIG. 1 a schematic illustration of an embodiment of a device 10 (e.g., a biosensor monitoring device) which may be employed in accordance with various aspects of the present invention. The device 10 includes a base 12 and a body 14 which may be employed for insertion into a subject (e.g., a fruit, such as a grape, an animal, a human being, etc) in order to monitor biometrical data, such as pH, sugar content (e.g., sucrose, glucose), analytes, etc. The base 12 has a circuit assembly 18 and an assembly 19 which cooperates with the circuit assembly 18 to monitor biometrical data. Assembly 19 includes a capacitor plate 20, an impermeable layer 32 supporting a conductive layer 22, a hydrogel 24 (a swelling polymer), a dielectric 36 (e.g., air) between the conductive layer 22 and the capacitor plate 20, and a filter (or permeable membrane) 48. The body 14 is bound to or connects with the base 12, and includes an aperture 40 wherethrough bio-organism fluid may flow to enter a capillary tube 44 which terminates in the filter 48 for filtering and/or allowing the passage of the bio-organism fluid into contact with the hydrogel 24. The hydrogel 24 has a chemical agent, such as acrylic acid, which is uniquely responsive to a chemical characteristic (e.g., pH or sugar) in the bio-organism fluid. When the hydrogel 24 comes in contact with the bio-organism fluid, the chemical agent uniquely responds to the chemical characteristic and causes the hydrogel to respond accordingly, such as by swelling. The chemical agent is tailored to the particular chemical characteristic. Thus, the particular chemical agent will depend on the particular chemical characteristic which is to be monitored and/or measured, and will change in accordance with the desired chemical characteristic.

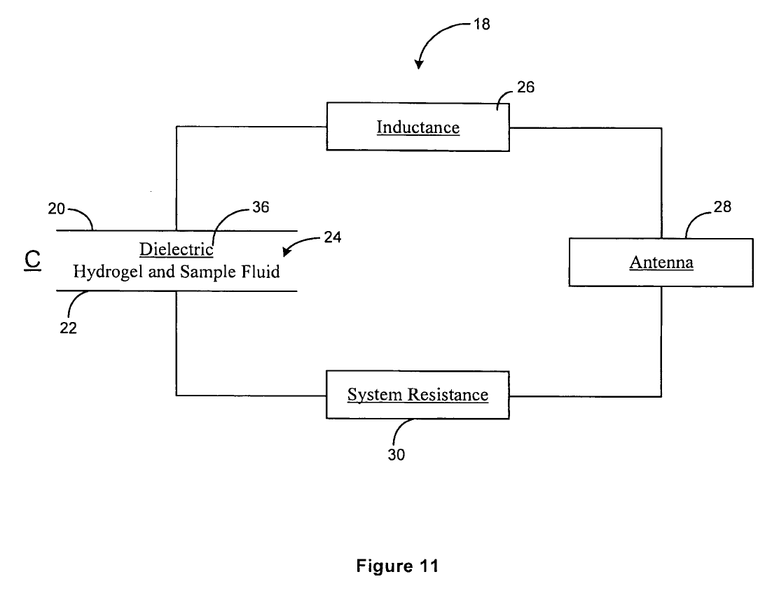

The circuit assembly 18, as best shown in FIG. 11, includes an inductance 26 connected in series with capacitor plate 20, an antenna 28 connected in series with the inductance 26 to produce a resonance frequency (i.e., a change in resonance frequency) in accordance with a change in capacitance which is directly related to the measurand (i.e., the chemical characteristic) in the bio-organism fluid, and parasitic resistance 30 connected in series with the antenna 28 and the conductive layer 22 (or capacitor plate).

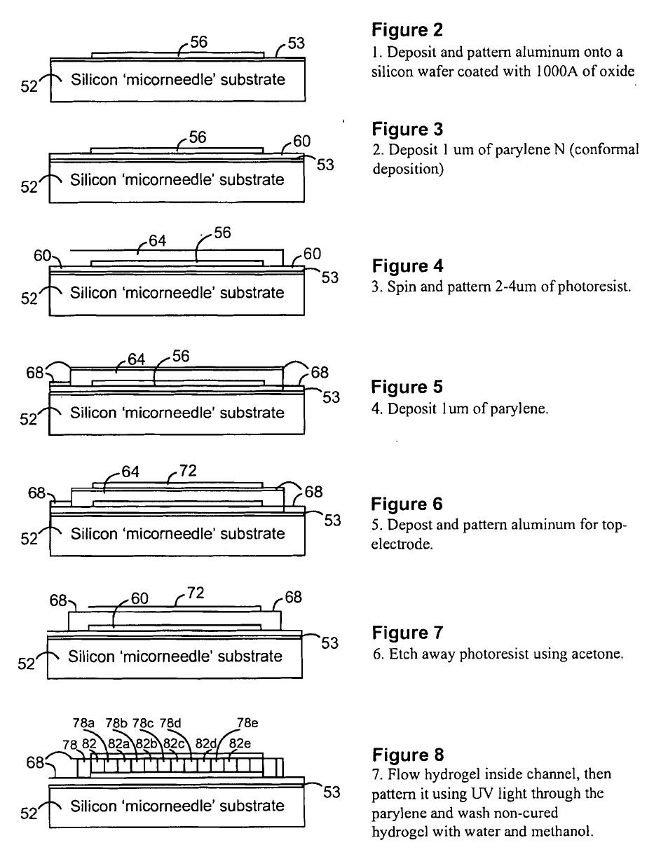

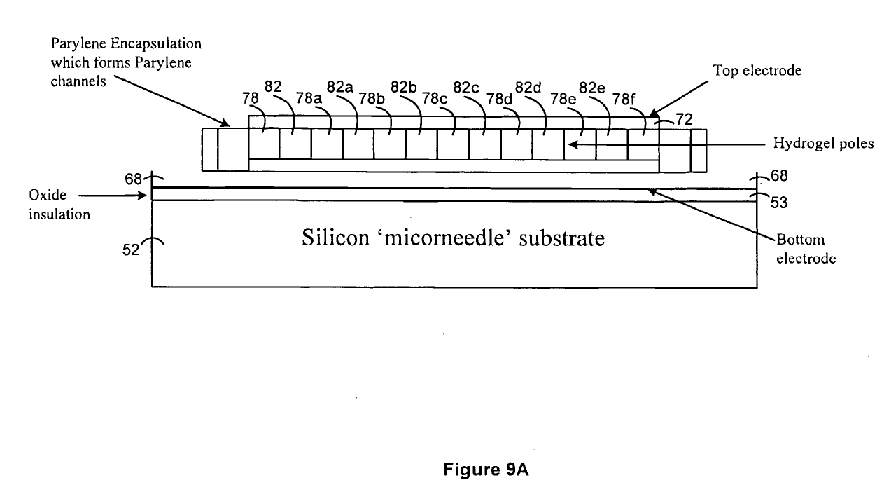

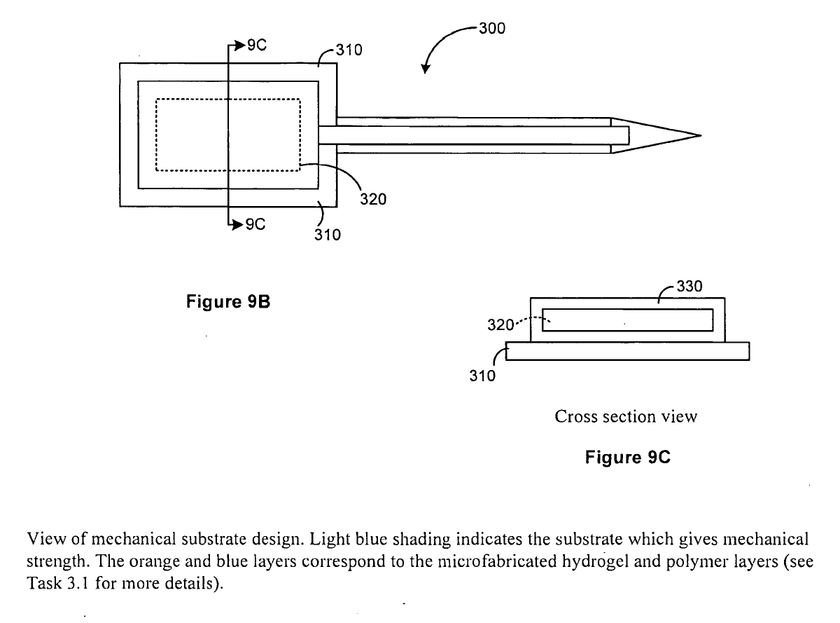

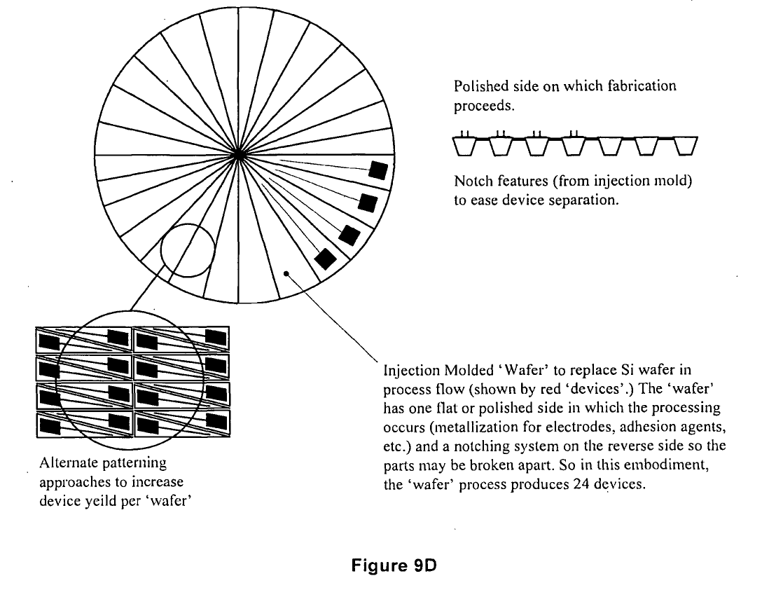

Referring now to FIGS. 2-9A for another embodiment of the present invention, there is seen a schematic flow diagram for producing another embodiment of the capacitor/hydrogel assembly wherein there is a plurality of channels (e.g., parylene channels) wherethrough bio-organism fluid may flow to come in contact with a plurality of hydrogel poles. In response to change in a chemical characteristic, such as pH or glucose, the hydrogel poles swell in every direction, particularly in a vertical direction. The swelling induces a change in the space or gap between the two electrodes (or capacitor plates), which may be measured in accordance with a change in capacitance. In FIG. 2 a substrate assembly is illustrated as aluminum 56 having been deposited and patterned onto a silicon wafer substrate 52 coated with 1000 A of oxide insulation however other substrate materials such as glass or plastic may be used 53. FIG. 3 illustrates the substrate assembly after conformally depositing 1 μm of parylene N 60 on the oxide insulation 53. FIG. 4 illustrates a side elevation view of the substrate assembly after spinning and patterning 2-4 μm of photoresist 64. FIG. 5 illustrates a side elevation view of the substrate assembly after depositing 1 um of parylene N 68 on top of the photoresist 64 and parylene N 60. FIG. 6 is a side elevational view of the substrate assembly after depositing and patterning any conductive material 72 (e.g., aluminum) for top-electrode. FIG. 7 is a side elevational view of the substrate assembly after etching away the photoresist 64 using acetone. FIG. 8 is a side elevational view of the finally produced substrate assembly after flowing hydrogel through the inside channel within the parylene N 68 and after subsequently patterning the hydrogel using UV light through the parylene N 68 and washing non-cured hydrogel with water and methanol. FIG. 9A is an exploded view of the view in FIG. 8. As best shown in FIGS. 8-9A, the produced hydrogel, generally illustrated as 78, has a plurality of hydrogel posts 78a, 78b, 78c, 78d, 78e, and 78f, separated by channels, generally illustrated as 82 and more specifically including channels 82a, 82b, 82c, 82d, and 82e.

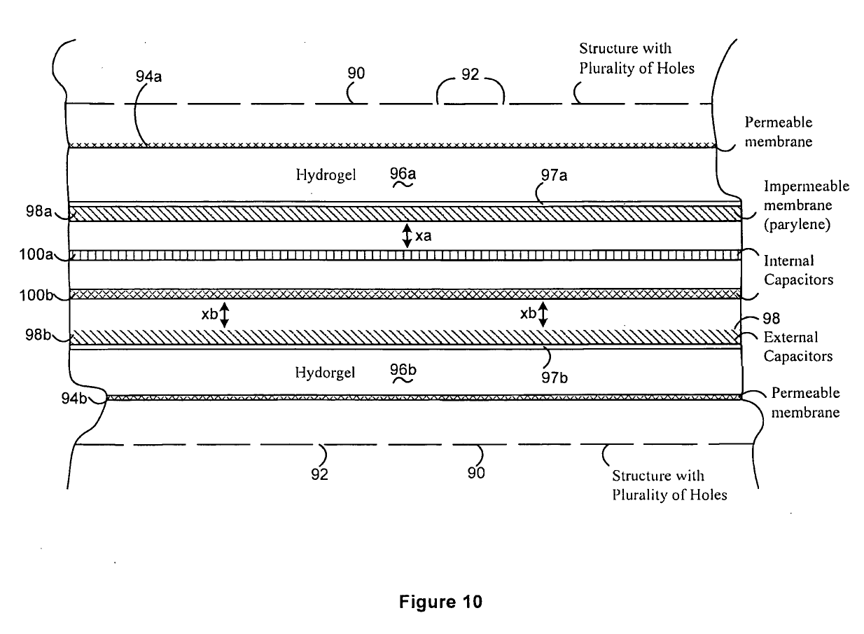

Referring now to FIG. 10 there is seen another embodiment of the hydrogel/capacitors assembly for detecting a change in capacitance which is directly related to the measurand in the bio-organism fluid. In FIG. 10 there is seen a structure 90 having a plurality of apertures 92 where through bio-organism fluid flows to come in contact with hydrogels 96a and 96b after respectively passing through permeable membranes 94a and 94b, respectively. Hydrogels 96a and 96b respectively expand against impermeable membranes (parylene membranes) 97a and 97b. This respectively causes the distance xₐ between external capacitor plate 98a and internal capacitor plate 100a and the distance x_{b} between external capacitor plate 98b and internal capacitor plate 100b to change along with the corresponding respective capacitance values. As will be further explained below, a change in chemical characteristic (e.g., pH) within the bio-organism may be found in accordance with the determined change in distances (and capacitance values) between capacitor plates. Because there are two distances (i.e., xₐ and x_{b}) whose changes are being monitored and/or determined, an average of these two may be obtained for determining an average change in corresponding respective capacitance values, thus determining an average change in chemical characteristics. For some embodiments of the invention, this could produce a more accurate monitoring of biometrical data.

The hydrogel (e.g., hydrogel 24, or hydrogel 78) used in implementations of various embodiments of the present invention may be any suitable changing or swelling chemical (e.g., polymer) or hydrogel, depending on the particular chemical characteristic which is to be monitored. In a preferred embodiment of the present invention, the hydrogel includes a mixture of acrylic acid and 2-hydroxyethyl methacrylate (in a 1:4 molar ratio), ethylene glycol dimethacrylate (1 wt %) and a photoinitiator (3 wt %). Acrylic acid is the chemical agent which gives the sensitivity to pH (the chemical characteristic) changes. For various embodiments, the hydrogel swells for high pH (>7) and shrinks at low pH (<4). 2-hydroxyethyl methacrylate (HEMA) is the monomer forming the chains into the hydrogel, and ethylene glycol dimethacrylate (EGDMA) is the crosslinking agent. As previously indicated in referencing FIGS. 2-9A, once the liquid has been mixed, it may be poured onto the substrate and exposed to UV light (wave length is 365 nm) through a photo-mask, for example. The curing time depends on the intensity of the UV light, the monomer mixture and the photoinitiator. Once the photopolymerization is done, the remaining liquid may be flushed using methanol and DI water.

For various embodiments of the present invention, this hydrogel is a preferred hydrogel for several reasons. First, it is compatible with microfabrication techniques. Like photoresist, it can be spun on a substrate and patterned. Further, the feature size can be as low as 50 μm, which is sufficient for many applications in accordance with embodiments. Another feature of hydrogels is that they have a good shelf life. A non-photopolymerized mixture can be stored at room temperature for three to four months. A photopolymerized pattern of hydrogel can be stored at room temperature for two to three weeks. It does not need to be hydrated, which means that it is compatible with the deposition process of parylene (at room temperature but under vacuum).

The hydrogel can enable transduction of a biochemical signal (diffusion of an analyte through its matrix) into a mechanical signal (swelling of the hydrogel). However, in accordance with embodiments, another transducer for transforming this mechanical signal into an electric signal can also be included. There are numerous ways to transduce a mechanical displacement into an electric signal. The swelling of the hydrogel may change the capacitance between two plates of a capacitor in accordance with embodiments. Alternatively, conversion of this motion into an electric signal can be done using either: (i) the motion of a piece of magnetic material in and out of a magnetic circuit to induce a change in inductance; (ii) the displacement of a diaphragm to change the resonance frequency of a resonator; (iii) changing the resistance of a resistor; and/or (iv) any suitable method that can be utilized with pressure sensors or the like.

For various embodiments of the invention it should be understood that the analyte may be measured with hydrogel between two electrodes (as way to correct for a capacitance change of the solution itself), or where the swelling of the hydrogel moves one electrode and the gap between the other electrode is held in air/vacuum. No hydrogel between electrodes.

Direct chemical to electrical transduction mechanisms can also be measured with this device. In some particular analytes, the capacitance of the hydrogel layer will be altered as function of the measurand. In this case, the capacitance of the hydrogel can be measured between two electrodes. This capacitance can be compared to that of an air filled electrode pair where the upper electrode is driven by the swelling process. The combination of capacitance between these two electrode pairs can be used to measure or cancel the chemical response.

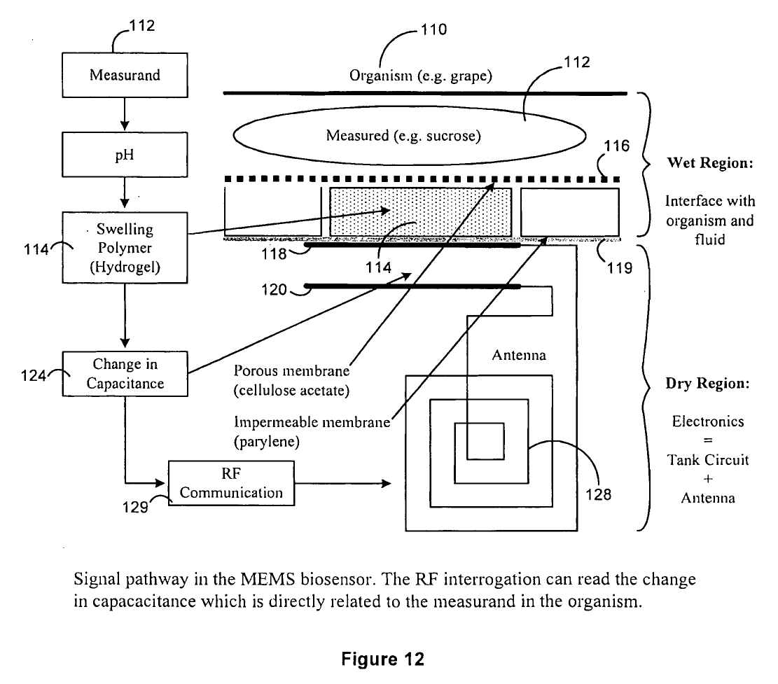

Referring now to FIG. 12 there is seen a block flow diagram for an embodiment of the signal pathway, along with a diagram of the antenna coupled to the hydrogel/capacitors assembly for detecting a change in capacitance which is directly related to the measurand in the organism fluid. As shown, an embodiment of the invention includes a wet region and a dry region. The wet region includes contacting a bio-organism fluid (such as juice from a grape) having a measurand 112, such as sucrose, with the swelling polymer (hydrogel 114) after passing through permeable membrane 116. The wet region also includes impermeable, flexible membrane 119. The dry region includes spaced capacitor plates 118 and 120 whose capacitance changes (i.e., change in capacitance 124) when capacitor plate 118 moves toward capacitor plate 120. The dry region also includes RF communicator (antenna 128) for producing an RF communication 129.

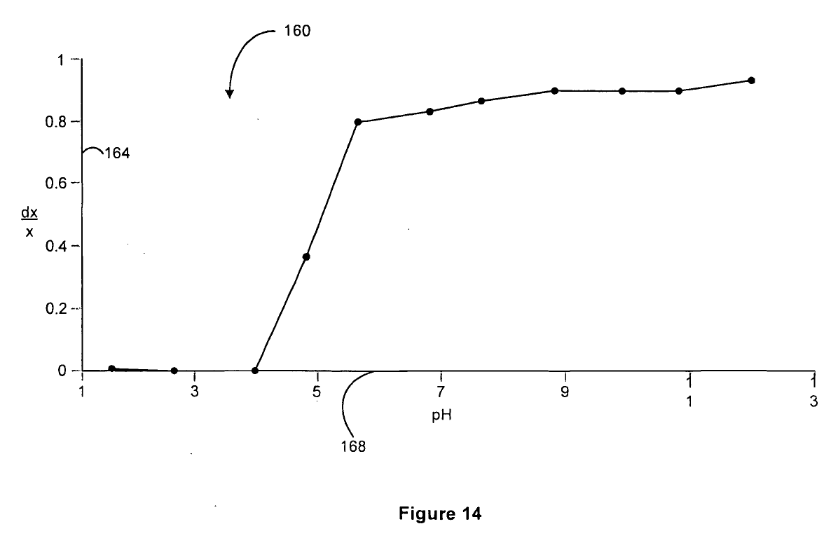

It is to be understood that because embodiments of the present invention relate to changes in various chemical characteristic (e.g., ΔpH) or analytes, the biosensor monitoring device for various embodiments of the present invention needs to be initially calibrated; more specifically, it is necessary to initially calibrate the biosensor monitoring device response to the particular chemical characteristic or analyte being monitored and/or measured. In some embodiments, the change in analyte value is the relevant data (such as sugar concentration reaching a saturation condition, local maxima or minima (ΔpH=0) in value) in which case initial calibration to establish a particular output value to a given measurand may not be required. Calibration is not only a function of the geometry and design of the biosensor monitoring device, but also the hydrogel composition, particularly since the hydrogel has a chemical agent that is responsive to a particular chemical characteristic of the biological matter, the bio-organism fluid. More particularly, it is necessary to initially produce a standard response or swelling curve, such as that shown in FIG. 14.

FIG. 14 is a graph of dx/x (change in distance between capacitor plates/initial distance between capacitor plates before the change in distance) vs. pH (i.e., the pH on the x-axis for any particular dx/x on the y-axis). The recipe for the hydrogel in producing the graph of FIG. 14 was: acrylic acid and 2-hydroxyethyl methacrylate (in a 1:4 molar ratio), ethylene glycol dimethacrylate (1 wt %) and a photoinitiator (3 wt %). The photoinitiator was DMPA (alpha-dimethoxy-phenylacetophenone). To produce such a graph, the biosensor monitoring device is to be put into contact with a series of sample bio-organism fluid having a known pH or known analyte concentration (the temperature during this measurement needs to be constant). This series of measurements allows the production of a standard response or swelling curve for the biosensor monitoring device. A standard response or swelling curve is a curve of swelling function (e.g., Δx/x) on the y-axis vs. the particular monitored chemical characteristic (e.g., pH) or monitored concentration of the analyte (e.g., sucrose, etc) on the x-axis. Once this is done, one can use the biosensor monitoring device to measure and/or monitor unknown pH, or unknown concentrations of analytes, in a bio-organism fluid from a subject, since one can back calculate the pH or the concentration by using the amount of hydrogel swelling measured by the biosensor monitoring device and the standard response or swelling curve which was initially prepared.

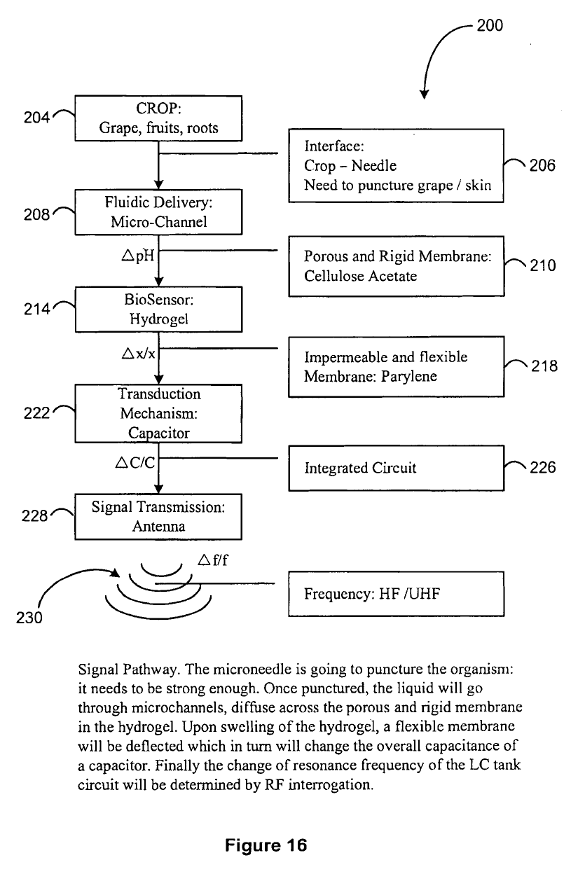

Referring now to FIG. 16 there is seen a block flow diagram for another embodiment of a signal pathway, illustrating the production of a change in resonance frequency (Δf/f) in accordance with the particular measurand in the bio-organism fluid being monitored and/or measured. A microneedle 206 punctures an organism 204 (a crop, such as grape, fruits, roots, etc) for fluidic delivery 208 of a bio-organism fluid into micro-channels. The bio-organism fluid passes through a porous and rigid membrane 210 (i.e., cellulose acetate membrane) and comes in contact with biosensor 214 (i.e., the hydrogel) for determining a ΔpH (the monitored biometrical data). The biosensor 214 is located between the porous, rigid membrane 210 and the impermeable, flexible membrane 218 and transforms a chemical signal into a mechanical signal. Swelling of the biosensor 214 causes a transduction; that is, a change in distances (Δx/x) between capacitor plates and a corresponding change in capacitance (ΔC/C) of the capacitor plates (electrodes). The integrated circuit 226 (e.g., such as circuit in FIG. 11) includes an antenna which transmits a signal transmission 228 which may be represented as Δf/f (i.e., frequency UF/UHF). Thus, once the microneedle punctures a subject or organism, bio-organic fluid will pass through microchannels, diffuse across the porous, rigid membrane and into the biosensor. Upon swelling of the biosensor, the flexible membrane will be deflected, which in turn will change the overall capacitance of a capacitor. Finally, the change in resonance frequency of the LC tank circuit will be determined by RF interrogation.

The RF interrogation should encompass a band of frequencies within the range of expected output values. The initial resonant frequency of the device, as given by:

$${f}_{o} = \frac{1}{2 π \sqrt{LC}}$$

establishes the initial output frequency. As the capacitance is altered by the aforementioned transduction mechanisms, the new device resonant frequency is related to the value of the measurand. So far as the initial and as measured frequencies fall within the range of the interrogator circuitry/reader and the measured frequencies are related to individual sensors, there is no requirement on the production of a known resonant frequency. Moreover, since the reader can measure a change in frequency, Δf, or an absolute frequency, there is no need for an initial calibration.

In one exemplary MIB design in accordance with embodiments, several key values can be determined. First, a measurement of a change in pH can be considered (e.g., ΔpH as imposed by industry standards). The change of pH can induce a swelling of the hydrogel which is equivalent to a displacement: Δx/x, which depends on the hydrogel sensitivity, the flexible membranes, etc. The membrane displacement can induce a change of capacitance, so ΔC/C can be considered. Finally, the change of capacitance may correlate to the change of resonance frequency of the LC tank circuit: Δf/f may be imposed by the characteristics of the device which interrogates the LC tank circuit.

Aspects of embodiments can include the miniaturization, encapsulation and integration of the hydrogel and the transduction mechanism into a microneedle or a device which could be inserted into a bio-organism. In one exemplary design in accordance with embodiments, the hydrogel may fill the reservoir of an in-plane microneedle. Part of the microneedle wall can be made with a porous material, such as porous silicon or cellulose acetate. The swelling of the hydrogel may be detected upon change of the capacitance of two capacitor plates placed alongside of the hydrogel. Advantages of this design include: (i) the response time will be relatively fast since the hydrogel is exposed to fluid in every direction; and (ii) due to geometric constraint, the swelling of the hydrogel may be greatly amplified, adding to the sensitivity of the device. In a second exemplary design, out-of-plane microneedles may be used. Such a design might resemble the signal pathway, as discussed above, since the different components would be stacked.

In a third exemplary design, the capillary structure is formed by the electrode, insulator and hydrogel and the substrate is shaped so as to improve the structural integrity of the device in order to puncture the organism of interest. In a fourth exemplary design, no such microneedles are required so far as the analyte is in contact with the transduction mechanism. FIGS. 9B and 9C illustrate another embodiment of the device, a microneedle generally illustrated as 300. A substrate 310 supports an encapsulator 330 which encapsulates the hydrogel/capacitors assembly 320. The encapsulator 330 may be formed from any suitable substance, such as plastic. The microneedle 300 will be used to puncture the skin of a subject, such as a human being, an animal, or a fruit (e.g., a grape). After puncture bio-organism fluid (e.g., interstitial fluid) of the subject will be drawn by capillary force, into the sensor allowing its diffusion into the hydrogel and subsequent volumetric change. FIG. 9D illustrates a “wafer” process for producing a multiplicity of devices.

The packaging and encapsulation of the hydrogel, as well as the encapsulation of the electronic circuit components so that they are not harmed by the wet chemistry of the hydrogel or the bio-fluids, is another aspect of embodiments of the present invention. In order to convert a chemical signal (e.g., pH or concentration of glucose) into a mechanical displacement, two different membranes may be used. One may be a porous but rigid membrane designed to allow diffusion of the fluid into the hydrogel. This membrane should preferably be substantially rigid to maximize the deflection of the second membrane, but porous enough so that fluid diffuses through the membrane into the hydrogel as fast as possible. Another membrane may be a non-porous but flexible membrane and its function may be to separate the wet region (e.g., hydrogel and microfluidic channels) from the dry region (e.g., integrated circuit and antenna). This membrane may also be designed to transduce the swelling of the hydrogel. Therefore, the membrane should be strong enough so that it does not tear or break, but flexible enough so that it can transduce most of the volume change.

In one implementation, cellulose acetate and parylene may be chosen as the materials for these two membranes. Cellulose acetate is widely used in the pharmaceutical industry for its properties as a filter. Cellulose acetate has been shown to be porous and rigid and has been used in a MEMS water-powered drug delivery system. Membranes of cellulose acetate can be fabricated through the mixing of 15% cellulose acetate and two solvents, 33% ethanol and 52% acetone, for example. The polymer solution may then be coated on a substrate and then dried at room temperature for about 30 seconds. Afterward, the solution may be immersed in a water quench bath also at room temperature for about 30 minutes and finally dried at room temperature for about a day. The fabricated white color membrane can be hot embossed to create features at the microscale. Parylene is a widely used polymer in the fields of biosensor and microtechnology. Since the deposition of parylene is typically carried out at room temperature and the layer formed is very conformal, parylene can be used at the end of a process to cover the device with a thin layer and serve as a protective barrier layer. Moreover, the hydrogel can be fully dried and then rehydrated without losing its properties, so the hydrogel is compatible with the parylene deposition process carried out at room temperature and under low vacuum (10 to 100 mTorr). As it is known to be biocompatible, this final layer would allow for the use of microneedles in contact with any biological organisms.

According to embodiments, in order to sense a position of the deflected membrane, a series resonant circuit (e.g., formed by connecting an inductor in series with the MEMS capacitor) may be used. The resulting circuit is commonly referred to as an LC tank oscillator circuit. The MEMS capacitor is the parallel combination of the actuator capacitor and associated parasitic capacitances. Depending on the particular implementation, parasitic capacitances can be the hydrogel, the cellulose acetate and/or just air. To take into consideration the losses in the inductor and MEMS capacitor, a resistor may be added into the model circuit. Accordingly, one design challenge is to correctly evaluate this system resistance. Furthermore, the integration of an antenna introduces additional impedance, capacitance and resistance to the device at the system level. As these values are not affected by the analyte or transduction mechanism, these values can be varied to optimize system performance for the various embodiments, ie. To maximize device range, maximize signal to noise, adjust resonant frequency (center frequency range), etc.

At the electrical resonance frequency, inductive and capacitive reactances cancel each other and the resonance frequency can be expressed as:

$${f}_{o} = \frac{1}{2 π \sqrt{{LC}_{MEMS}}} \qquad ( 1 )$$

The quality factor, Q, of such a circuit can be expressed in terms of the inductance (L), capacitance (C_{MEMS}) and resistance (R):

$$Q = \frac{1}{R} \sqrt{\frac{L}{C}} \qquad ( 2 )$$

Some simple calculations demonstrate the method by which the system can be developed and evaluate key design requirements. For example, the required sensitivity in the wine industry for a hand-held portable pH-meter is ΔpH=0.05. Using the standard swelling curve in FIG. 14, for pH-sensitive hydrogels, this ΔpH corresponds to a Δx/x equals to 0.02. Assuming that the characteristic length of the hydrogel is 500 μm, then Δx=10 μm. For the capacitor, one suitable design may be 2 square plates separated by air. For this simple geometry, we have:

$$\frac{Δ C}{C} = \frac{Δ x}{x} = 0.02 \qquad ( 3 )$$

In the foregoing formula

$$C = {ɛ}_{o} {ɛ}_{r} \frac{A}{x} ,$$

where x is the distance, εₒ is the absolute permittivity and εᵣ is the relative permittivity of the dielectric, and A is the area of the capacitor plates. The value 0.02 comes from the standard curve of FIG. 14.
Finally, using equation (1), we find:

$$\frac{Δ f}{f} = \frac{Δ C}{2 C} = 0.01 \qquad ( 4 )$$

Simple computations show that if C is chosen to be around 10⁻¹³F and L around 10⁻⁴H, the resonant frequency is approximately 170 MHz and Δf is 2 MHz. Accordingly, given typical design constraints, the inductance may be the parameter adjusted in some embodiments. It is to be understood that these values may significantly change based on system resistance and/or RFID detector frequency. Further, in some applications, analog and digital electronics may be integrated onto the same piece of silicon or substrate, such that the sensor can be interrogated using standard RFID frequency bands and protocols.

In some alternate embodiments of the present invention, a biometric data system may also be used to trip an alarm by making electrical contact at a given analyte value (e.g., 31 Brix). In this approach, hydrogel swelling as described above could cause a diaphragm buckling, which could result in electrical contact between electrodes.

Thus, practice of various embodiments of the present invention provides a micro electro-mechanical system (MEMS) radio-frequency (RF)-Interrogated Biosensor (MIB) device and associated method. The device is advantageously a wireless, powerless, communicating device which may sense changes in a subject's characteristic. For example, a measurement of an analyte, such as pH, glucose, penicillin and/or antibodies (or any pH related changes) may be sensed and transferred via a network to a device for analysis, storage or other processing. The subject may be a plant or crop, organism, animal, a human being, etc.

Practice of various embodiments of the present invention provides a micro electro-mechanical system (MEMS) radio-frequency (RF)-Interrogated Biosensor (MIB) device which may be employed to puncture the skin of a fruit and transmit data about the glucose content in the fruit using RF-interrogation. The MIB device includes a microfabricated structure that comprises a microneedle, a functionalized gel, a tuned tank oscillator circuit, and a miniature antenna. Other embodiments can use different designs including omitting, adding or modifying components described herein. For example, in different designs a needle, capillary, tube or other conduit or conveyance might be substituted for the microneedle. In some designs such a conduit may not be needed as where the characteristic being sensed is in contact with, or sufficiently close to, the MIB without needing conveying.

Practice of various embodiments of the present invention utilizes volume changes in the functionalized polymer (hydrogel) in order to change the tuning of the simple tank oscillator circuit. The transduction of a chemical to an electric signal may be done through the use of micro-fabrication, screen printing, or alternate means. Such functionalized polymers (hydrogels) have been developed to be sensitive to a wide variety of analytes (glucose, penicillin and antibodies) and can be treated similarly to photoresist with similar application. Curing procedures using UV light can be easily implemented in a microfabricated process, for example. The hydrogel changes in volume in response to changes in the biological organism in which the microneedle is inserted. Other transduction approaches are contemplated in accordance with embodiments, such as changes in the dielectric constant, changes in voltage and/or change in leakage current, etc.

Changes in the tank oscillator circuit can be determined by remote RF interrogation, using a passive scheme in which the need for a local power source on the MIB is made unnecessary. The glucose level (Brix) in a grape may measured using a refractometer. For an embodiment, the particular exemplary grape application requires the grape to be crushed onto the device. For other embodiments of the present invention one could insert the MIB into grapes and read the glucose level via a remote RF interrogation. The MIB can enable collection of data on a substantially continuous basis. Further, these measurements can be read using a handheld reader. In one embodiment, a multivalued variable includes identification (“ID”) and other information associated with a person, animal or crop being monitored. The variable value or values are recovered by wireless communication such as with a radio frequency (RF) receiver. Other types of data recovery are possible, such as with infrared, acoustic, microwave, electrical leads. In general, any suitable communication link can be used.

Practice of various embodiments of the present invention provides a micro electro-mechanical system (MEMS) radio-frequency (RF)-Interrogated Biosensor (MIB) device which could form a mesh of biosensor integrated in the proposed network. In one embodiment, the device may be passive (in order to eliminate the power consumption problem), so the device can be detected by measuring the amount of RF energy that is attenuated by the tank oscillator circuit in the MIB. Thus, in some embodiments, frequency-dependent attenuation of the RF energy between the transmitter/receiver antenna pair can be initially measured. Subsequently, supplemental time-domain detection may be used to discriminate between multiple devices. In this fashion, the MIB device may transmit some biological signal about the organism in which it is inserted. Typical measurands may be pH or glucose concentration in the fluid found in the organism being monitored, such as a human, animal, etc. A variety of other biometric and/or chemical detection events could be performed (i.e. temperature, humidity, light intensity). In other designs where power is available, an active circuit can be used to transfer the data.

Practice of other embodiments of the present invention provides a micro electro-mechanical system (MEMS) radio-frequency (RF)-Interrogated Biosensor (MIB) device which could be inserted into fruits and/or other bio-organisms to monitor different analytes and transmit these data to a RF network. Embodiments could also be used for environmental and livestock monitoring. In one example application, embodiments could be used to allow monitoring of micro-climates in forests. Further, this type of device could also be adapted to human monitoring. For example, a MIB according to embodiments could be used to monitor and provide early alarm indication for at risk patients. In general, any type of subject and any characteristic of the subject can be the target of monitoring or sensing.

Functionalized polymers or hydrogels, or even an osmotic driving agent, such as a functionalized salt, are a well-characterized and developed class of polymer with a high affinity for water. They swell as water diffuses through the polymer fiber matrix. They can be controlled by pH and utilized to actuate the variable capacitor in the MIB. They exhibit many characteristics that are critical in a material integrated in a MEMS biosensor. Hydrogels have been developed to swell in response to specific conditions such as temperature, electric field, and concentration of different analytes. These functionalized hydrogels can be engineered to respond to specific ranges of analytes and to exhibit specific volume swelling ratios. However, the characteristic which makes hydrogels very attractive for MEMS biosensor applications in accordance with embodiments is the integration of the hydrogels into a microfabrication process. The hydrogels can be relatively easily applied to a substrate and patterned like a photoresist, with a feature size compatible with microtechnology. The curing wavelength for a hydrogel used in implementations is about 365 nm, substantially identical to I-line photoresist, and obviating the need for specialty processing equipment.

The microneedles may be configured for use with embodiments of the present invention. The microneedles may be hollow and are fabricated of polysilicon. The in-plane microneedles are 100 μm wide, 100 μm thick but actually 5 millimeters long. The thickness of the needle wall is just 3 μm. For the out-of-plane design, they are 200 μm long and 200 μm wide at the basis. The asymmetric design allows a better penetration of the skin of the bio-organism. In one embodiment, a plastic injection molding process can be used to make such microneedles. For example, a silicon chip may be used as a mold. As another example, a sensor can be integrated with a silicon chip and the structure may be packaged in plastic using an injection molding process. Using an injection molding process for the microneedles or other portions of the system can allow for reduced size and/or production expense. In yet another embodiment, the capillary structure is fabricated on top of microfabrication compatible substrates (Silicon, glass, plastic, metal).

In one application of the invention, a MIB (MEMS RF-Interrogated Biosensor) in accordance with embodiments can allow remote measurements to be made of the glucose level (Brix) in a grape on the vine. In order for this sensor to require no on-board power source or battery, an energy-efficient signal pathway may be used. The signal pathway is the means by which a chemical detection made by the sensor, is transformed into an electric signal that can be transmitted over the RF-link to the rest of the communication network.

Practice of various embodiments of the present invention provides a micro electro-mechanical system (MEMS) radio-frequency (RF)-Interrogated Biosensor (MIB) device employing a signal pathway between the measurand (measured in the organism) and the RF signal. One component in this biosensor, which can transform a chemical signal into a mechanical signal, is the functionalized swelling polymer located between the porous and the impermeable membranes. Such transforming of the membrane deflection into a change of an electric resistance and/or capacitance is well known in the art for MEMS pressure sensors and MEMS implantable biosensor applications.

In one exemplary MIB design in accordance with embodiments, several key values can be determined. First, a measurement of a change in pH can be considered (e.g., ΔpH as imposed by industry standards). The change of pH can induce a swelling of the hydrogel which is equivalent to a displacement: Δx/x, which depends on the hydrogel sensitivity, the flexible membranes, etc. The membrane displacement can induce a change of capacitance, so ΔC/C can be considered. Finally, the change of capacitance may correlate to the change of resonance frequency of the LC tank circuit: Δf/f may be imposed by the characteristics of the device which interrogates the LC tank circuit.

Aspects of embodiments can include the miniaturization, encapsulation and integration of the hydrogel and the transduction mechanism into a microneedle or a device which could be inserted into a bio-organism. In one exemplary design in accordance with embodiments, the hydrogel may fill the reservoir of an in-plane microneedle. Part of the microneedle wall can be made with a porous material, such as porous silicon or cellulose acetate. The swelling of the hydrogel may be detected upon change of the capacitance of two capacitor plates placed alongside of the hydrogel. Advantages of this design include: (i) the response time will be relatively fast since the hydrogel is exposed to fluid in every direction; and (ii) due to geometric constraint, the swelling of the hydrogel may be greatly amplified, adding to the sensitivity of the device. In a second exemplary design, out-of-plane microneedles may be used. Such a design might resemble the signal pathway, as discussed above, since the different components would be stacked.

According to embodiments, a biosensing device may integrate a hydrogel into a microneedle. The microneedle can be used to puncture the most superficial part of the bio-organism's skin in order to probe only the biological fluid, for example. Aspects of embodiments include the integration of a functionalized hydrogel and a wireless and powerless (or power-scavenged) communication circuit onto a microneedle. Since the analyzed fluid is may be a biological fluid, it may be necessary to develop a filter system, such as a substantially rigid but porous membrane to protect the hydrogel from biological fluids and/or contamination by proteins, large and small molecules.

Although the invention has been described with reference to specific embodiments thereof, these embodiments are merely illustrative, and not restrictive of the invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

It will also be appreciated that one or more of the elements depicted in the drawings/figures can also be implemented in a more separated or integrated manner, or even removed or rendered as inoperable in certain cases, as is useful in accordance with a particular application.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims.

What is claimed is:

1. A method for obtaining data of a subject, comprising:
Inserting or immersing a sensor into the subject and retrieving fluid from the subject;
transforming a chemical signal from the fluid by using a functionalized polymer;
detecting a change in the functionalized polymer; and
transmitting an indication of the change in the functionalized polymer.

2. The method of claim 1, wherein the functionalized polymer includes a swelling polymer.

3. The method of claim 2, further comprising:
using a capacitive plate to detect a change in the swelling polymer; and
determining a resonant frequency related to a position of the capacitor plate.

4. The method of claim 1, wherein the data includes biometrics data.

5. The method of claim 4, wherein the biometrics data includes a pH level.

6. The method of claim 2, wherein the swelling polymer includes a hydrogel.

7. The method of claim 3, further comprising:
providing radio-frequency (RF) signals to determine the resonant frequency.

8. An apparatus for obtaining data of a subject, the apparatus comprising:
means for inserting or immersing a sensor into the subject and retrieving fluid from the subject;
means for transforming a chemical signal from the fluid by using a swelling polymer;
means for detecting a change in the swelling polymer; and
means for transmitting an indication of the change in the swelling polymer.

9. An apparatus for determining biometrics data of a living organism, the apparatus comprising:
a capillary tube configured to receive fluid from the living organism;
a hydrogel solution having a volume responsive to one or more characteristics of the fluid;
a capacitor having at least one plate responsive to the volume of the hydrogel solution; and
an inductor coupled to the capacitor, the inductor and the capacitor forming a circuit having a resonant frequency.

10. The apparatus of claim 9, wherein the resonant frequency is related to
a pH of the fluid.

11. The apparatus of claim 9, wherein the living organism includes a grape.

12. The apparatus of claim 9, further including an antenna.

13. The apparatus of claim 9, further including a filter coupled to the capillary tube.

14. A method for monitoring biometrical data comprising: providing a chemical matter having a chemical agent; contacting the chemical matter with a biological matter; detecting a change in the chemical matter to produce a signal; altering the signal into an electrical signal; and obtaining biometrical data from the electrical signal.

15. The method of claim 14 wherein said chemical agent is sensitive to a chemical characteristic, and the biological matter includes the chemical characteristic such that monitoring of biometrical data includes determining a change in the chemical characteristic.
